(19) United States
(12) Patent Application Publication
(10) Pub. No.: US 2017/0158999 A1
(43) Pub. Date: Jun. 8, 2017

(54) MODULAR BIOREACTOR FOR CULTURE OF BIOPAPER BASED TISSUES

(71) Applicant: The Government of the United States of America, as represented by the Secretary of the Navy, Arlington, VA (US)

(72) Inventors: Russell Kirk Pirlo, Fort Washington, MD (US); Peter K. Wu, Ashland, OR (US); Bradley R. Ringeisen, Lorton, VA (US)

(73) Assignee: The Government of the United States of America, as represented by the Secretary of the Navy, Arlington, VA (US)

(21) Appl. No.: 15/367,890

(22) Filed: Dec. 2, 2016

Related U.S. Application Data
(60) Provisional application No. 62/262,635, filed on Dec. 3, 2015.

(51) Int. Cl. C12M 1/12; C12M 1/34; C12M 1/00

(52) U.S. Cl. CPC C12M 25/14; C12M 25/06; C12M 23/22; C12M 41/46

(57) ABSTRACT

An apparatus having: an enclosure having a base and a top and a plate. The base and the top each have an interior surface which together define the interior of the enclosure. The base and the top each have an inlet fluid channel and an outlet fluid channel from the interior of the enclosure to the exterior of the enclosure. The plate is in the interior of the enclosure and has a frame having an opening, a gasket, and a biopaper spanning the opening. The plate divides the interior of the enclosure into two cavities. A portion of the biopaper is not touching the frame, the gasket, or the interior surfaces. The biopaper is fluid communication with the fluid channels.

This application claims the benefit of U.S. Provisional Application No. 62/262,635, filed on Dec. 3, 2015. The provisional application and all other publications and patent documents referred to throughout this nonprovisional application are incorporated herein by reference.

### TECHNICAL FIELD

The present disclosure is generally related to bioreactors for tissue engineering

### DESCRIPTION OF RELATED ART

One standard research tool employed in tissue engineering and biological research is the transwell membrane. These membranes are used in multiwall plates and allow culturing single and bi layers of cells with media on each side. Other methods use perfused chambers where cells or tissues are cultured on the bottom or sides of a chamber through which media is flowed. Neither of these methods allow for stacking of multiple bilayers of cells.

### BRIEF SUMMARY

Disclosed herein is an apparatus comprising: an enclosure comprising a base and a top and a plate. The base and the top each comprise an interior surface which together define the interior of the enclosure. The base and the top each comprise an inlet fluid channel and an outlet fluid channel from the interior of the enclosure to the exterior of the enclosure. The plate is in the interior of the enclosure and comprises a frame having an opening, a gasket, and a biopaper spanning the opening. The plate divides the interior of the enclosure into two cavities. A portion of the biopaper is not touching the frame, the gasket, or the interior surfaces. The biopaper is fluid communication with the fluid channels.

### BRIEF DESCRIPTION OF THE DRAWINGS

A more complete appreciation will be readily obtained by reference to the following Description of the Example Embodiments and the accompanying drawings.

FIG. 1 schematically illustrates an embodiment of the bioreactor having one plate.

FIG. 2 schematically illustrates a plate as viewed from above.

FIG. 3 shows another embodiment in which two plates create an inter-plate cavity.

FIG. 4 shows an exploded view of a configuration having electrodes.

FIG. 5 shows an exploded view of another embodiment.

FIG. 6 shows the addition of a bubble catch chamber.

### DETAILED DESCRIPTION OF EXAMPLE EMBODIMENTS

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

Disclosed herein is a modular bioreactor based around stackable inserts that serve as a substrate for various culture conditions. Different tissues will require different environmental conditions including air or media exposure, as well as different cell and biomaterial arrangements. The modular system supports various configurations such as monolayer cultures and bilayer cultures with cells on either side of a single insert. Multi-layer cultures with multiple inserts having mono or bilayer cultures each as well as some inserts having the entire inter-insert space filled by hydrogel or other cell matrix/scaffolding components.

The bioreactor may be used as a platform for constructing, culturing, and studying engineered tissues. The platform is modular in that it is an assembly of pieces which can be altered to allow for the creation of specific tissue construct, culture, or monitoring or other research scenarios. Central to all configurations is a plate or insert which is the foundation of the smallest tissue block supported by the platform. The plate is a framed biopaper, a single membranous layer upon or into which single or multiple cell types can be applied via traditional or cell printing methods. The bioreactor allows for any number of these biopaper supported tissue layer inserts to be stacked, either directly or with spacers to create vacancies between layers. Perfusion of each layer is facilitated by holes and channels built into the biopaper frame, which align with fluidic channels in the bioreactor. All this is to create a flexible in vitro platform for tissue engineering and research.

The plates or inserts are framed with a rigid material that allows them to fit into a receiving area on the inside of the bioreactor, pins, rails, or other geometric fittings align the insert into a specific area in the bioreactor. This alignment allows for the insert to be coupled to microfluidic channels in the bioreactor as well as aligned to other similar inserts which can be stacked above or below. This alignment of features between layers can be used to create complex three-dimensional cell/biomaterial/environmental arrangements using any number of conventional two-dimensional cell and bio factor printing techniques. The insert may be removable with or without disassembly of the bioreactor.

The inserts may be separated by a polymer gasket or spacer that not only serves to seal the chamber, but also to create inter-insert spaces which can be filled with cell culture media, extracellular matrix components (hydrogel) and cell components.

Inserts may be biopapers such as those disclosed in U.S. Pat. No. 8,669,086, US Pat. Appl. Pub. No. 2014/0154771, and U.S. patent application Ser. No. ______ entitled “BIOPAPERS AS A SUBSTRATE FOR TISSUE CULTURE, filed by Pirlo et al. on the same day as the present application, metal or polymer frames with membranes overlaid or electrospun onto them, and may support tissue constructs including cell monolayers, bilayers, 3D hydrogels and 3D cell/hydrogel/scaffolding composites. Biopapers can be used that are degradable or non-degradable, and that have mechanical and chemical characteristics that are selected to suit the cells and tissues being cultured. The biopapers can also include electrodes.

The frames of the biopaper may also include channels which act as connecting conduits between fluid channels in the bioreactor and fluid channel/vascular constructs/media spaces in the insert, including any cell structures created on it or in the attached 3D cell/hydrogel/scaffolding composite.

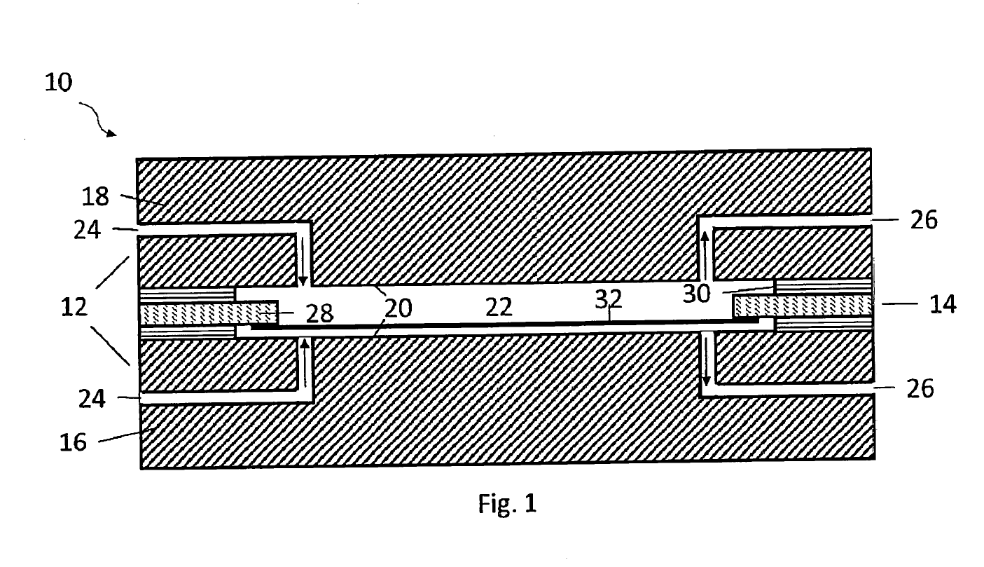

FIG. 1 schematically illustrates an embodiment of the bioreactor having one plate. The bioreactor 10 includes two main components: an enclosure 12 and a plate 14. The enclosure 12 may be made of any material that is compatible with the biomaterials and fluids to be used in the bioreactor. Biocompatible polymers that are inert, non-leaching, and able to withstand autoclaving, such as polyoxymethylene (—CH₂—O—), may be used. The enclosure 12 includes a base 16 and a top 18, which may be separable from and attachable to each other using any compatible fasteners, such as screws. When the base and the top are placed together and/or attached together, each comprises an interior surface 20 that faces the interior surface of the other, together defining the interior 22 of the enclosure. The base 16 and the top 18 each comprise an inlet fluid channel 24 and an outlet fluid channel 26 from the interior 22 of the enclosure to the exterior. The designation of inlet and outlet may be arbitrary, as generally a fluid may pass through the bioreactor in either direction.

The bioreactor may include windows in the top and/or the bottom over the interior, bleed chamber, inlets, or outlets for performing optic-based sensing. Optical or fluorescent methods may include sensing coupons for pH or O₂.

The plate 14 divides the interior of the enclosure into two cavities on either side of the plate. The plate 14 comprises a frame 28 having an opening, a gasket 30, and a biopaper 32 spanning the opening. The biopaper 32 is positioned to be in fluid communication with the fluid channels 24, 26, that is, fluid entering each of the inlet channels may contact one side or other of the biopaper, then exit the enclosure through the outlet channels. A portion of the biopaper 32 is not touching the frame 28, the gasket 30, or the interior surfaces 20 so that fluid may contact that portion on both sides of the biopaper 32. The frame 28 and the gasket 30 may be positioned to prevent any fluid flow directly between the cavities other than through the biopaper itself, if possible. The frame 28 and the gasket 30 may be separate components are may be a unitary component.

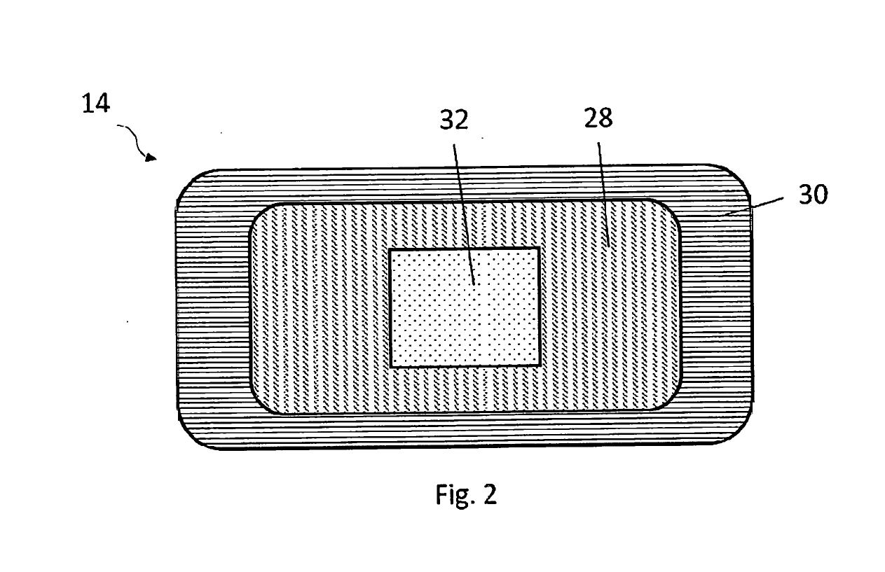

The frame may be made of similar compatible materials as the enclosure. One suitable frame material is Cyclic Olefin Copolymer (COC, e.g. ethylene-norbornene copolymer). COC has a glass transition temperature that can be selected to allow for hot embossing of micro channels for perfusion of the supported tissue layer, but resist melting when autoclave sterilized. The gasket is also a compatible material, and also prevents fluid flow into or out of the enclosure. Polytetrafluoroethylene is one suitable gasket material. FIG. 2 schematically illustrates a plate 14 as viewed from above.

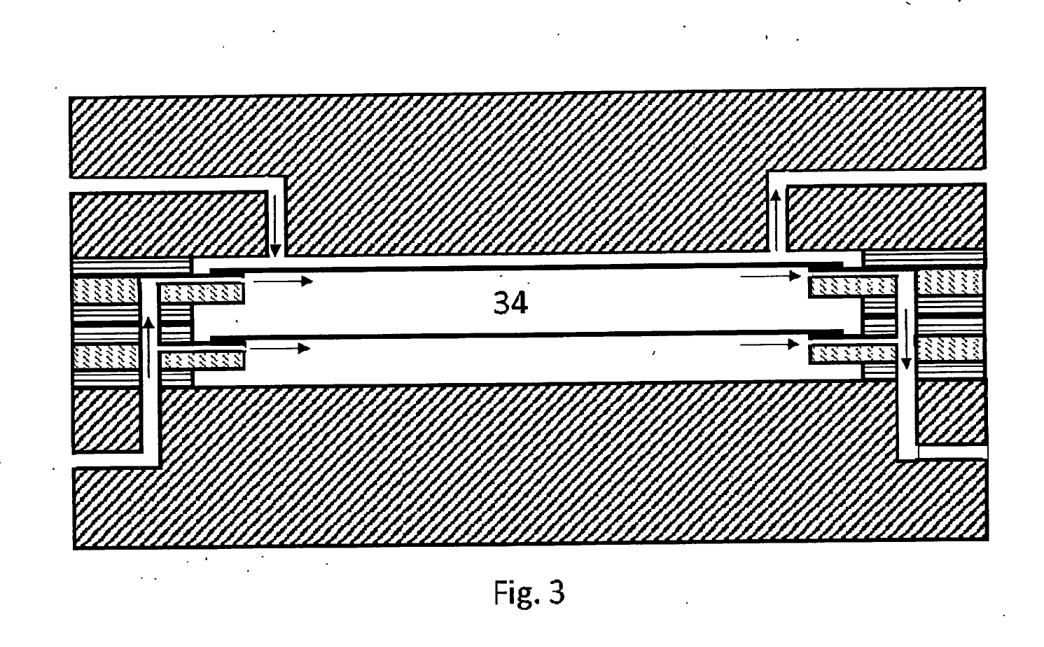

FIG. 3 shows another embodiment in which two plates create an inter-plate cavity 34 between the two plates in the interior in addition to the two cavities discussed above. The drawing also shows that the frame and/or gasket holding the biopaper may also contain grooves, channels, and/or clear through holes that when stacked create fluidic channels and branches, allowing for a single tissue construct to be perfused at multiple points throughout, in a fashion that scales with each layer. The two inserts may be used to create a multilayer tissue construct where a first media flows through the lower two isolated cavities, and a second media (such as air) is flowed through the top most cavity. This example uses one insert with clear through holes, and one with no clear through holes. Alternatively, each of the three cavities may have its own inlet and outlet. Even more plates may be stacked in the bioreactor to create more inter-plate cavities.

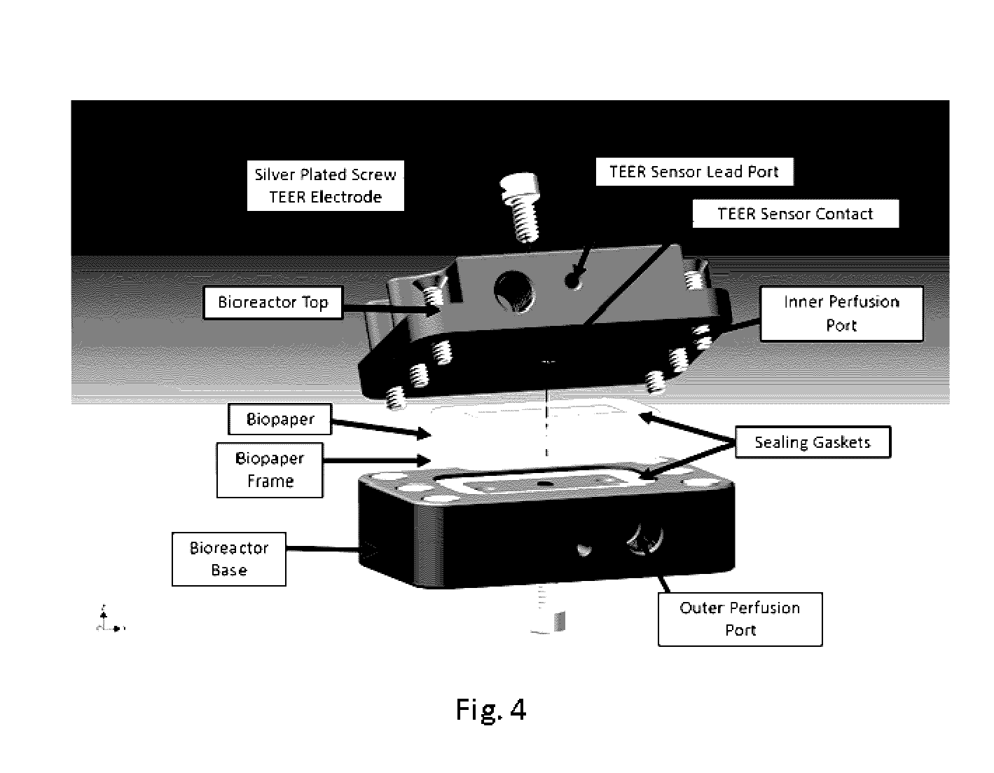

Further components of the bioreactor may include a pair of electrodes in the base and top that are in fluid communication with either side of the biopaper. These electrodes may allow for trans-membrane electrical resistance (or trans endothelial/epithelial electrical resistance (TEER)) to be measured non-invasively and with no movement of the electrodes. These electrodes may comprise silver. FIG. 4 shows an exploded view of such a configuration. This design may be used to support the culture of blood-brain barrier (BBB) tissue.

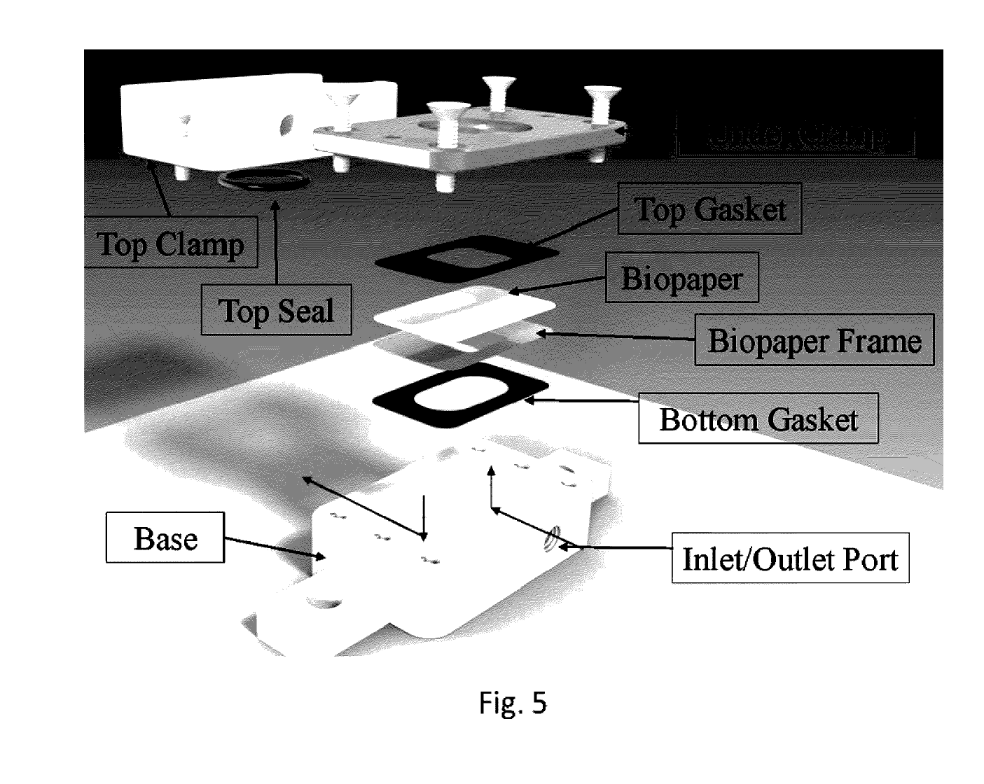

FIG. 5 shows an exploded view of another embodiment in which an under clamp is used, allowing the bioreactor top to be removed. This configuration of the bioreactor allows for accessing the tissue without complete disassembly. It is useful for observation, probing, and exposing a tissue to aerosolized test agents. This configuration may be used to culture lung tissue with an air/media interface.

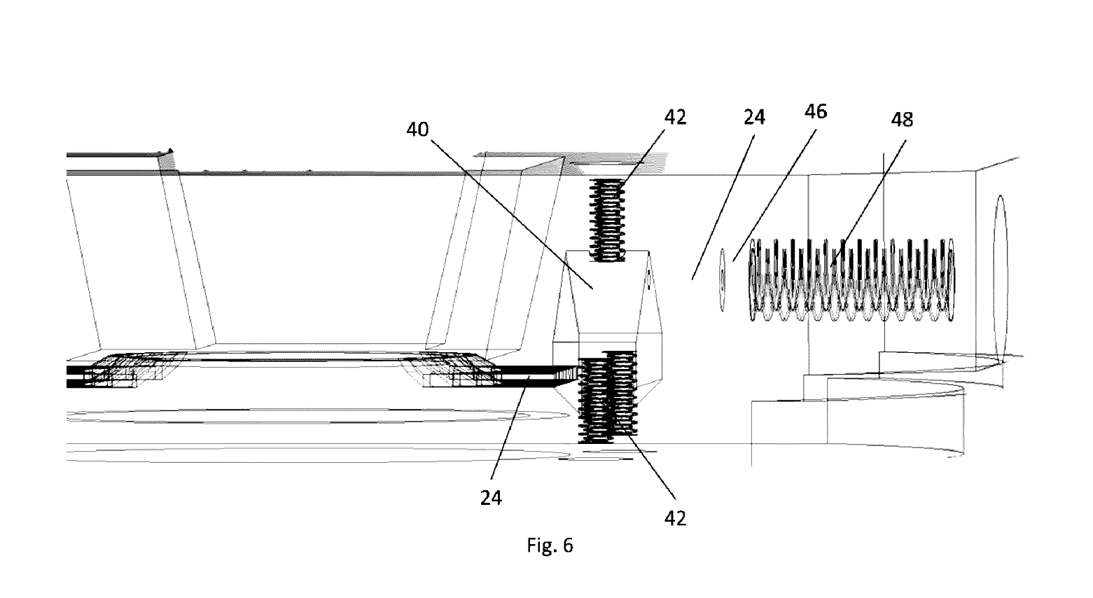

FIG. 6 shows the addition of a bubble catch chamber 40. The tapered top of the bubble chamber 40 is above the perfusion/inlet channels 24 and forces bubbles to and through the bleed screw 42 because they float. Because of height differentials in the bleed chamber top/bottom and the perfusion channels, bubbles and or drops are trapped at the top or bottom of the chamber and can be blead/drained by removal/loosening of bleed screws 44. The tapers and bleed screws 44 on the bottom work the same when the bioreactor is inverted. Because access to the other bleed screw is restricted when the bioreactor is assembled, it can also be used to drain fluid when air is the perfused medium. The design may include a reusable polymer septum 46 as the inlet/outlet ports so that standard syringe needles may be used as the connecting pieces. Hollow threaded screws 48 may be used to create a seal of the inlet outlet septa into the bioreactor.

The system may further include electronics as part of the biopaper frame/biopaper plate. These electronics may allow for sensing and stimulation activity to be performed at the biopaper surfaces.

The system can be adapted to model specific tissues or create various modeling scenarios by altering the material used in the membrane component of the biopaper, as well as any other components that are applied to the biopaper. The bioreactor platform can be adapted to thick or thin tissue models by including different numbers and/or types of biopaper inserts. When the bioreactor is used to construct thick, solid, tissue constructs, by stacking multiple cell laden biopaper inserts, it may have the benefit of pre-maturation of individual layers before stacking (either in dish or in the multiple bioreactors before stacking). This can solve a long standing problem of necrosis developing in the center of thick tissue constructs where diffusional transport of oxygen, nutrients, and waste are insufficient. With this bioreactor design each layer can have a mature engineered vascular/fluidic system before stacking which aligns perfectly to the flow channels of the bioreactor frame of the biopaper insert inserted into the matched recess in the bioreactor. The perfusion system described is easily scalable to any number of inserts as through holes in the rigid frame act as the main flow conduit, and open faced channels in the rigid frame become individual fluidic branches of the main tissue as they are stacked.

The system can be used to create thin tissue constructs that model barrier tissues such as lung or the blood brain barrier where the cavities on either side of a membrane are isolated from each other via the biopaper membrane and the surrounding bioreactor and gaskets. This isolation provides for modeling barrier tissues with differing apical and basal media components, or different phases, such as liquid media and air.

The electrodes allow for continuous, highly reproducible, and non-invasive monitoring of TEER, a significant improvement over conventional TEER apparatus that have a user positioned electrode and require opening of the tissue culture environment for reading.

The rigid frame stacking system allows for high resolution alignment of layers to each other which allows for three dimensional patterns of cells and materials (biological or otherwise) to be created via two dimensional patterning and printing methods.

The rectangular form shown is used to aid in the subtractive machining method used, but other shapes could be used if the bioreactor were formed by additive or molding methods, however the basic design of a stack of framed membranes which create a fluidic manifold for perfusion of any number of layers via stacking would be retained. Size (area and depth) of chambers may be adjusted to account for desired tissue volumes, and required media/air reservoir spaces.

Obviously, many modifications and variations are possible in light of the above teachings. It is therefore to be understood that the claimed subject matter may be practiced otherwise than as specifically described. Any reference to claim elements in the singular, e.g., using the articles “a”, “an”, “the”, or “said” is not construed as limiting the element to the singular.

What is claimed is:

1. An apparatus comprising:
an enclosure comprising a base and a top;
wherein the base and the top each comprise an interior surface which together define the interior of the enclosure; and
wherein the base and the top each comprise an inlet fluid channel and an outlet fluid channel from the interior of the enclosure to the exterior of the enclosure; and
a plate in the interior of the enclosure comprising a frame having an opening, a gasket, and a biopaper spanning the opening;
wherein the plate divides the interior of the enclosure into two cavities;
wherein a portion of the biopaper is not touching the frame, the gasket, or the interior surfaces; and
wherein the biopaper is fluid communication with the fluid channels.

2. The apparatus of claim 1, wherein the frame and the gasket prevent fluid flow between the cavities other than through the biopaper.

3. The apparatus of claim 1, wherein the plate comprises a plate flow channel though the frame, the gasket, or both.

4. The apparatus of claim 1, wherein the apparatus comprises two or more of the plates creating one or more inter-plate cavities in the interior of the enclosure.

5. The apparatus of claim 1, wherein the biopaper has living cells deposited on one or both sides of the biopaper.

6. The apparatus of claim 5, wherein each side of the biopaper has a different type of living cell deposited thereon.

7. A method comprising:
providing the apparatus of claim 1;
flowing a first fluid into the top inlet flow channel and out of the top outlet flow channel; and
flowing a second fluid into the base inlet flow channel and out of the base outlet flow channel.

8. The method of claim 7, wherein providing the apparatus comprises:
depositing living cells on one or both sides of the biopaper; and
placing the plate into the enclosure.

9. The method of claim 7, further comprising:
removing the plate from the enclosure; and
examining any biomaterial on the biopaper.

10. The method of claim 7, wherein the first fluid or the second fluid is air.

11. The method of claim 7, wherein the first fluid, the second fluid, or both is a liquid that promotes growth of the cells.

12. The apparatus of claim 1, further comprising:
a base electrode passing through the base and in fluid communication with the biopaper; and
a top electrode passing through the top and in fluid communication with the biopaper.

13. The apparatus of claim 12, wherein the base electrode and the top electrode comprise silver.

14. A method comprising:
providing the apparatus of claim 12;
flowing a first fluid into the top inlet flow channel and out of the top outlet flow channel;
flowing a second fluid into the base inlet flow channel and out of the base outlet flow channel; and
monitoring the resistance between the base electrode and the top electrode.

15. The apparatus of claim 1, further comprising:
a gas/liquid separator that removes gas or liquid from a fluid entering the top inlet fluid channel or the bottom inlet fluid channel before the fluid contacts the biopaper.

16. The apparatus of claim 1, further comprising:
a septum in the fluid path of the top inlet fluid channel or the bottom inlet fluid channel.

17. The apparatus of claim 1, further comprising:
An electronic circuit incorporated into the frame.

18. The apparatus of claim 1, wherein the enclosure comprises one or more optically transparent windows.
